SEQUENCE LISTING (2 sequences)

SEQ ID NO: 1 - protein, 85 aa, Artificial Sequence
  MAGE Core signature sequence - consensus
LIXVLXXIXXXGXXAPEEXIWEXLXXMXXXXGXEXXXXGXPXXLLTXXXVQEXYLXYXQVPXSXPXXYEFLWGPRAXXETXXXKV

SEQ ID NO: 2 - protein, 450 aa, artificial sequence
  fusion protein of Lipoprotein D fragment, Mage3 fragment, and histidine tail
MDPKTLALSLLAAGVLAGCSSHSSNMANTQMKSDKIIIAHRGASGYLPEHTLESKALAFAQQADYLEQDLAMTKDGRLVVIHDHFLDGLTDVAKKFPHRHRKDGRYYVIDFTLKEIQSLEMTENFETMDLEQRSQHCKPEEGLEARGEALGLVGAQAPATEEQEAASSSSTLVEVTLGEVPAAESPDPPQSPQGASSLPTTMNYPLWSQSYEDSSNQEEEGPSTFPDLESEFQAALSRKVAELVHFLLLKYRAREPVTKAEMLGSVVGNWQYFFPVIFSKASSSLQLVFGIELMEVDPIGHLYIFATCLGLSYDGLLGDNQIMPKAGLLIIVLAIIAREGDCAPEEKIWEELSVLEVFEGREDSILGDPKKLLTQHFVQENYLEYRQVPGSDPACYEFLWGPRALVETSYVKVLHHMVKISGGPHISYPPLHEWVLREGEEGGHHHHHHH

(19) United States
(12) Patent Application Publication
(10) Pub. No.: US 2015/0147350 A1
(43) Pub. Date: May 28, 2015

(54) Method Of Treating Cancer With MEGEA3 Immunotherapeutic With BRAF Inhibitor And/Or MEK Inhibitor

(71) Applicant: GlaxoSmithKline LLC, Wilmington, DE (US)

(72) Inventors: Catherine Marie Ghislaine Gerard, Rixensart (BE); Sylvie Laquerre, King of Prussia, PA (US); Peter F. Lebowitz, King of Prussia, PA (US); Frederic Francois Eugene Lehmann, Rixensart (BE); Jamila Louahed, Rixensart (BE)

(21) Appl. No.: 14/364,770

(22) PCT Filed: Dec. 19, 2012
(86) PCT No.: PCT/US12/70582

§ 371 Date: Jun. 12, 2014

Related U.S. Application Data
(60) Provisional application No. 61/578,943, filed on Dec. 22, 2011.
(60) Provisional application No. 61/579,028, filed on Dec. 22, 2011.

(51) Int. Cl. A61K 39/00; A61K 31/506; A61K 31/519; A61K 38/16

(52) U.S. Cl. CPC A61K 39/0011; A61K 38/164; A61K 31/506; A61K 31/519
USPC 424/192.1

(57) ABSTRACT

A combination of anti-neoplastic agents that provides increased activity over monotherapy, or in some cases at least an unexpected lack of negative interaction. In particular, the drug combination that includes a MAGE-A3 immunotherapeutic, in combination with a B-Raf inhibitor, particularly N-{3-[5-(2-Amino-4-pyrimidinyl)-2-(1,1-dimethylethyl)-1,3-thiazol-4-yl]-2-fluorophenyl}-2,6-difluorobenzenesulfonamide, or a pharmaceutically acceptable salt thereof, and/or a MEK inhibitor, particularly N-{3-[3-cyclopropyl-5-(2-fluoro-4-iodo-phenylamino)6,8-dimethy; -2,4,7-trioxo-3,4,6,7-tetrahydro-2H-pyrido[4,3-d]pyrimidin-1-yl]phenyl}acetamide, or a pharmaceutically acceptable salt or solvate thereof is described.

### FIELD OF THE INVENTION

The present invention relates to a method of treating cancer in a mammal and to combinations useful in such treatment. In particular, the method relates to a novel combination comprising a MAGEA3 immunotherapeutic, with either a B-Raf inhibitor, particularly N-{3-[5-(2-Amino-4-pyrimidinyl)-2-(1,1-dimethylethyl)-1,3-thiazol-4-yl]-2-fluorophenyl}-2,6-difluorobenzenesulfonamide, or a pharmaceutically acceptable salt thereof, and/or a MEK inhibitor, particularly N-{3-[3-cyclopropyl-5-(2-fluoro-4-iodo-phenylamino)6,8-dimethy; -2,4,7-trioxo-3,4,6,7-tetrahydro-2H-pyrido[4,3-d]pyrimidin-1-yl]phenyl}acetamide, or a pharmaceutically acceptable salt or solvate thereof, pharmaceutical compositions comprising the same and methods of using such combinations and compositions in the treatment of conditions in which the inhibition of B-Raf and the raising of a MAGEA3 specific immune response is beneficial, eg. cancer.

### BACKGROUND OF THE INVENTION

Effective treatment of hyperproliferative disorders including cancer is a continuing goal in the oncology field. Generally, cancer results from the deregulation of the normal processes that control cell division, differentiation and apoptotic cell death and is characterized by the proliferation of malignant cells which have the potential for unlimited growth, local expansion and systemic metastasis. Deregulation of normal processes include abnormalities in signal transduction pathways and response to factors which differ from those found in normal cells.

Recently, B-Raf inhibitors have been investigated for use in treating cancer due to increased understanding of the Ras-Raf-MEK-ERK kinase pathway (known as the MAPK pathway). Particularly, the understanding that activation of Ras proteins in response to growth factors, hormones, cytokines, etc. stimulates phosphorylation and activation of Raf kinases which, in turn, phosphorylate and activate the MEK1 and MEK2 kinases which then phosphorylate and activate the ERK1 and 2 kinases.

Mutations in the MAPK substituent kinases are believed to negatively affect the growth signal functionality of the pathway, resulting in the establishment, development, and progression of a wide range of human cancers. Naturally occurring mutations in the B-Raf kinase have been observed in significant percentages of human melanomas (Davies, H., et al., Nature (2002) 9:1-6; Garnett, M. J. & Marais, R., Cancer Cell (2004) 6:313-319) and thyroid cancers (Cohen et al J. Nat. Cancer Inst. (2003) 95(8) 625-627 and Kimura et al Cancer Res. (2003) 63(7) 1454-1457), as well as at lower, but still significant, frequencies a number of other cancers.

Also recently, MEK inhibitors have been studied for use in cancer treatment. Mitogen-activated protein (MAP) Kinase/extracellular signal-regulated kinase (ERK) kinase (hereinafter referred to as MEK) is known to be involved in the regulation of cell proliferation as a kinase that mediates Raf-MEK-ERK signal transduction pathway, and the Raf family (B-Raf, C-Raf etc.) activates the MEK family (MEK-1, MEK-2 etc.) and the MEK family activates the ERK family (ERK-1 and ERK-2). Activation of Raf-MEK-ERK signal transduction pathway in cancer, particularly colorectal cancer, pancreatic cancer, lung cancer, breast cancer and the like, has been frequently observed.

In addition, since the signals produced by signal molecules such as growth factor, cytokine and the like converge to the activation of MEK-ERK, inhibitors of these functions are considered to more effectively suppress Raf-MEK-ERK signal transduction than suppression of the function of upstream kinases such as RTK, Ras, and Raf.

Moreover, it is also known that a compound having MEK inhibitory activity effectively induces inhibition of ERK1/2 activity and suppression of cell proliferation (The Journal of Biological Chemistry, vol. 276, No. 4, pp. 2686-2692, 2001), and the compound is expected to show effects on diseases caused by undesirable cell proliferation, such as tumor genesis and/or cancer.

Among possible cancer treatments other than kinase inhibitors, immunotherapeutic approaches targeting tumor antigens have shown promise. Among the different families of tumor antigens, the cancer/testis antigens (CT antigens) family has a particularly interesting pattern of expression. The MAGE-A3 gene belongs to this cancer/testis antigens, and belongs to a closely interrelated MAGE gene family, which is located on chromosome X and shares 64-85% identity in their coding sequences.

The corresponding MAGE-A3 protein is an antigen that was originally defined through its recognition by specific cytotoxic T lymphocytes (CTLs) on autologous melanoma cells (hence the term MAGE, for melanoma antigen). MAGE-A3 is immunogenic in humans, and several MAGE-A3-derived epitopes have been identified [Cancer Immunome Database 2010]. The MAGE-A3 gene is strictly tumor specific. It is not expressed in normal adult tissues, except in the testis, and it is expressed in embryonic tissues at some points in the fetal development or by trophoblastic cells of the placenta [DePlaen, 1994, De Smet, 1994, Takahashi, 1995, Chomez, 1996, De Smet, 1996], The gene is expressed in the cytoplasm of cells (i.e., spermatogonia in the testis and trophoblasts in the placenta) that do not bear HLA molecules on their surface [Haas, 1988, Boël, 1995, Chomez, 2001, Jungbluth, 2007]; therefore, MAGE-A3-derived epitopes cannot be presented to and recognized by CD8+ or CD4+ T cells. Consequently, immunization against the MAGE-A3 antigen is not expected to induce immune-related toxicities in humans and will result solely in an immune response against cancer cells that express MAGE-A3. MAGE-A3 is expressed in some percentages of many different tumors of different histological types including 65%; head and neck cancers, 65%; bladder cancer, 62%; hepatic cancer, 48%; esophageal cancer, 47%; NSCLC, 35%; ovarian cancer, 30%; leukemia, 29%; and prostate cancer, 18% [data from Van den Eynde, 1997].

Thus CT antigens, like MAGE-A3, are potential targets for specific cancer immunotherapy, as an immune response raised against the antigen should not affect normal tissues, but only cancer cells expressing the antigen. Tumor regression has been demonstrated in subjects with melanoma and bladder cancer by such CT antigen based immunotherapy (see e.g., Nishiyama et al., Immunotherapy of Bladder Cancer Using Autologous Dendritic Cells Pulsed with Human Lymphocyte Antigen-A24-specific MAGE-3 Peptide 2001, Clin. Cancer Res., 7:23-31; Thurner et al., Vaccination with Mage-3A1 Peptide-Pulsed Mature, Monocyte-Derived Dendritic Cells Expands Specific Cytotoxic T Cells and Induces Regression of Some Metastases in Advanced Stage IV Melanoma 1999, J. Exp. Med., 190:1669-1678; Coulie et al., A monoclonal cytolytic T-lymphocyte response observed in a melanoma patient vaccinated with a tumor-specific antigenic peptide encoded by gene MAGE-3, Proc Natl Acad Sci USA 98(18): 10290-10295 (2001)). More than 50 cancer/testis antigens have been described and, for many of them, specific epitopes recognized by T lymphocytes have been identified.

Though there have been many recent advances in the treatment of cancer with compounds such as the B-Raf inhibitors, MEK inhibitors, and antigens such as cancer/testis antigens, there remains a need for more effective and/or enhanced treatment of an individual suffering the effects of cancer.

### SUMMARY OF THE INVENTION

The present inventors have identified a combination of therapeutic agents that provides increased activity over monotherapy, or in some cases at least an unexpected lack of negative interaction. In particular, a method of treatment using the drug combination of a MAGEA3 immunotherapeutic, in combination with the B-Raf inhibitor N-{3-[5-(2-Amino-4-pyrimidinyl)-2-(1,1-dimethylethyl)-1,3-thiazol-4-yl]-2-fluorophenyl}-2,6-difluorobenzenesulfonamide or a pharmaceutically acceptable salt thereof and/or in combination with the MEK inhibitor: N-{3-[3-cyclopropyl-5-(2-fluoro-4-iodo-phenylamino)6,8-dimethy; -2,4,7-trioxo-3,4,6,7-tetrahydro-2H-pyrido[4,3-d]pyrimidin-1-yl]phenyl}acetamide, or a pharmaceutically acceptable salt or solvate thereof is described.

According to an aspect of the invention, there is provided a method of treating a susceptible cancer in a human in need thereof, said method comprising administering a combination comprising

(i) a MAGE-A3 immunotherapeutic, and
(ii) the B-Raf inhibitor represented by the structure of formula (I):

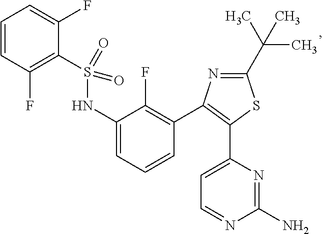 [SMILES: CC(C)(C)c1nc(-c2cccc(NS(=O)(=O)c3c(F)cccc3F)c2F)c(-c2ccnc(N)n2)s1]

or a pharmaceutically acceptable salt thereof (collectively referred to herein as “Compound A”).

According to an aspect of the invention, there is provided a method of treating a susceptible cancer in a human in need thereof, said method comprising administering a combination comprising

(i) a MAGE-A3 immunotherapeutic, and
(ii) the MEK inhibitor represented by structure (II):

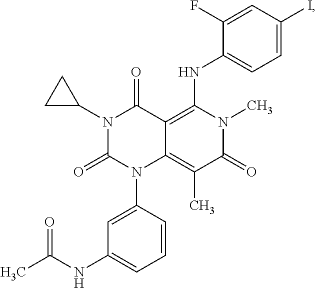 [SMILES: CC(=O)Nc1cccc(-n2c(=O)n(C3CC3)c(=O)c3c(Nc4ccc(I)cc4F)n(C)c(=O)c(C)c32)c1]

or a pharmaceutically acceptable salt or solvate thereof (collectively referred to herein as “Compound B”).

According to an aspect of the invention, there is provided a method of treating a susceptible cancer in a human in need thereof, said method comprising administering a combination comprising

(i) a MAGE-A3 immunotherapeutic,

### (ii) Compound A, and

(iii) Compound B.

According to another aspect of the invention, there is provided a method of treating a susceptible melanoma in a human in need thereof, said method comprising administering a combination comprising

(i) a MAGE-A3 immunotherapeutic, and

### (ii) Compound A.

According to another aspect of the invention, there is provided a method of treating a susceptible melanoma in a human in need thereof, said method comprising administering a combination comprising

(i) a MAGE-A3 immunotherapeutic, and

### (ii) Compound B.

According to another aspect of the invention, there is provided a method of treating a susceptible melanoma in a human in need thereof, said method comprising administering a combination comprising

(i) a MAGE-A3 immunotherapeutic,

### (ii) Compound A, and

(iii) Compound B.

According to another aspect of the invention, there is provided a method of treating a susceptible cancer in a human in need thereof, said method comprising administering a combination comprising

(i) a MAGE-A3 immunotherapeutic, and
(ii) N-{3-[5-(2-Amino-4-pyrimidinyl)-2-(1,1-dimethylethyl)-1,3-thiazol-4-yl]-2-fluorophenyl}-2,6-difluorobenzenesulfonamide methanesulfonate.

According to another aspect of the invention, there is provided a method of treating a susceptible cancer in a human in need thereof, said method comprising administering a combination comprising

(i) a MAGE-A3 immunotherapeutic, and
(ii) N-{3-[3-cyclopropyl-5-(2-fluoro-4-iodo-phenylamino)6,8-dimethy; -2,4,7-trioxo-3,4,6,7-tetrahydro-2H-pyrido[4,3-d]pyrimidin-1-yl]phenyl}acetamide dimethyl sulfoxide.

According to another aspect of the invention, there is provided a method of treating a susceptible cancer in a human in need thereof, said method comprising administering a combination comprising

(i) a MAGE-A3 immunotherapeutic, and
(ii) N-{3-[5-(2-Amino-4-pyrimidinyl)-2-(1,1-dimethylethyl)-1,3-thiazol-4-yl]-2-fluorophenyl}-2,6-difluorobenzenesulfonamide methanesulfonate, and
(iii) N-{3-[3-cyclopropyl-5-(2-fluoro-4-iodo-phenylamino)6,8-dimethy; -2,4,7-trioxo-3,4,6,7-tetrahydro-2H-pyrido[4,3-d]pyrimidin-1-yl]phenyl}acetamide dimethyl sulfoxide.

According to another aspect of the invention, there is provided a method of treating a susceptible cancer in a human in need thereof, said method comprising administering a combination comprising

(i) a MAGE-A3 immunotherapeutic, and
(ii) Compound A, together with a pharmaceutically acceptable diluents and/or carrier.

According to another aspect of the invention, there is provided a method of treating a susceptible cancer in a human in need thereof, said method comprising administering a combination comprising

(i) a MAGE-A3 immunotherapeutic, and
(ii) Compound B, together with a pharmaceutically acceptable diluents and/or carrier. According to another aspect of the invention, there is provided a method of treating a susceptible cancer in a human in need thereof, said method comprising administering a combination comprising
(i) a MAGE-A3 immunotherapeutic,
(ii) Compound A, together with a pharmaceutically acceptable diluents and/or carrier,
(iii) Compound B, together with a pharmaceutically acceptable diluents and/or carrier.

According to another aspect of the invention, there is provided a use of a combination comprising

(i) a MAGE-A3 immunotherapeutic, and
(ii) Compound A; for the treatment of a susceptible cancer in a human.

According to another aspect of the invention, there is provided a use of a combination comprising

(i) a MAGE-A3 immunotherapeutic, and
(ii) Compound B; for the treatment of a susceptible cancer in a human.

According to another aspect of the invention, there is provided a use of a combination comprising

(i) a MAGE-A3 immunotherapeutic,
(ii) Compound A; for the treatment of a susceptible cancer in a human, and
(iii) Compound B; for the treatment of a susceptible cancer in a human.

According to another aspect of the invention, there is provided a method of treating a susceptible cancer in a human in need thereof, said method comprising administering a combination comprising

(i) a MAGEA3 immunotherapeutic comprising a Protein D-MAGE-A3 fusion protein, and

### (ii) Compound A.

According to another aspect of the invention, there is provided a method of treating a susceptible cancer in a human in need thereof, said method comprising administering a combination comprising

(i) a MAGEA3 immunotherapeutic comprising a Protein D-MAGE-A3 fusion protein, and

### (ii) Compound B.

According to another aspect of the invention, there is provided a method of treating a susceptible cancer in a human in need thereof, said method comprising administering a combination comprising

(i) a MAGEA3 immunotherapeutic comprising a Protein D-MAGE-A3 fusion protein,

### (ii) Compound A, and

(iii) Compound B.

According to another aspect of the invention, there is provided a method of treating a susceptible melanoma in a human in need thereof, said method comprising administering a combination comprising

(i) a MAGEA3 immunotherapeutic comprising a Protein D-MAGE-A3 fusion protein, and

### (ii) Compound A.

According to another aspect of the invention, there is provided a method of treating a susceptible melanoma in a human in need thereof, said method comprising administering a combination comprising

(i) a MAGEA3 immunotherapeutic comprising a Protein D-MAGE-A3 fusion protein, and

### (ii) Compound B.

According to another aspect of the invention, there is provided a method of treating a susceptible melanoma in a human in need thereof, said method comprising administering a combination comprising

(i) a MAGEA3 immunotherapeutic comprising a Protein D-MAGE-A3 fusion protein,

### (ii) Compound A, and

(iii) Compound B.

According to another aspect of the invention, there is provided a method of treating a susceptible cancer in a human in need thereof, said method comprising administering a combination comprising

(i) a MAGEA3 immunotherapeutic comprising a Protein D-MAGE-A3 fusion protein, and
(ii) N-{3-[5-(2-Amino-4-pyrimidinyl)-2-(1,1-dimethylethyl)-1,3-thiazol-4-yl]-2-fluorophenyl}-2,6-difluorobenzenesulfonamide methanesulfonate.

According to another aspect of the invention, there is provided a method of treating a susceptible cancer in a human in need thereof, said method comprising administering a combination comprising

(i) a MAGEA3 immunotherapeutic comprising a Protein D-MAGE-A3 fusion protein, and
(ii) N-{3-[3-cyclopropyl-5-(2-fluoro-4-iodo-phenylamino)6,8-dimethy; -2,4,7-trioxo-3,4,6,7-tetrahydro-2H-pyrido[4,3-d]pyrimidin-1-yl]phenyl}acetamide dimethyl sulfoxide.

According to another aspect of the invention, there is provided a method of treating a susceptible cancer in a human in need thereof, said method comprising administering a combination comprising

(i) a MAGEA3 immunotherapeutic comprising a Protein D-MAGE-A3 fusion protein,
(ii) N-{3-[5-(2-Amino-4-pyrimidinyl)-2-(1,1-dimethylethyl)-1,3-thiazol-4-yl]-2-fluorophenyl}-2,6-difluorobenzenesulfonamide methanesulfonate, and
(iii) N-{3-[3-cyclopropyl-5-(2-fluoro-4-iodo-phenylamino)6,8-dimethy; -2,4,7-trioxo-3,4,6,7-tetrahydro-2H-pyrido[4,3-d]pyrimidin-1-yl]phenyl}acetamide dimethyl sulfoxide.

According to another aspect of the invention, there is provided a method of treating a susceptible cancer in a human in need thereof, said method comprising administering a combination comprising

(i) a MAGEA3 immunotherapeutic comprising a Protein D-MAGE-A3 fusion protein, and
(ii) Compound A, together with a pharmaceutically acceptable diluents and/or carrier.

According to another aspect of the invention, there is provided a method of treating a susceptible cancer in a human in need thereof, said method comprising administering a combination comprising

(i) a MAGEA3 immunotherapeutic comprising a Protein D-MAGE-A3 fusion protein, and
(ii) Compound B, together with a pharmaceutically acceptable diluents and/or carrier.

According to another aspect of the invention, there is provided a method of treating a susceptible cancer in a human in need thereof, said method comprising administering a combination comprising

(i) a MAGEA3 immunotherapeutic comprising a Protein D-MAGE-A3 fusion protein,
(ii) Compound A, together with a pharmaceutically acceptable diluents and/or carrier, and
(iii) Compound B, together with a pharmaceutically acceptable diluents and/or carrier.

According to another aspect of the invention, there is provided a use of a combination comprising

(i) a MAGEA3 immunotherapeutic comprising a Protein D-MAGE-A3 fusion protein, and
(ii) Compound A; for the treatment of a susceptible cancer in a human.

According to another aspect of the invention, there is provided a use of a combination comprising

(i) a MAGEA3 immunotherapeutic comprising a Protein D-MAGE-A3 fusion protein, and
(ii) Compound B; for the treatment of a susceptible cancer in a human.

According to another aspect of the invention, there is provided a use of a combination comprising

(i) a MAGEA3 immunotherapeutic comprising a Protein D-MAGE-A3 fusion protein,

### (ii) Compound A, and

(iii) Compound B, for the treatment of a susceptible cancer in a human.

In a further aspect of this invention is provided a method of treating cancer in a mammal in need thereof which comprises administering a therapeutically effective amount of a combination of the invention wherein the combination is administered within a specific period and for a duration of time.

### DESCRIPTION OF THE DRAWINGS

FIG. 1 shows the percentage of CD4+T cells producing cytokines in mice immunized twice with the MAGE-A3 ASCI+/−BRAF or MEK inhibitors

FIG. 2 shows the percentage of CD8+T cells producing cytokines in mice immunized twice with the MAGE-A3 ASCI+/−B-RAF or MEK inhibitors

FIG. 3 shows the mean titers of antibody response in mice immunized twice with MAGE-A3 ASCI+/−B-RAF or MEK inhibitors

### DETAILED DESCRIPTION OF THE INVENTION

### MAGE proteins and recMAGEA3

The cancer-testis antigen known as MAGE-A3 belongs to the MAGE-A sub-family which comprises 11 known members (MAGE-A 1-6 and 8-12). While other “MAGE-A” (melanoma antigen family A) genes have been reported (such as MAGE-A7, A13-15), the expression patterns of these genes suggest that they are pseudogenes (see e.g., Chomez et al., Cancer Research, 61:5544 (2001)). WO 94/23031 describes MAGE-A3. Human MAGE-A3 (also known as MAGE-3, MAGEA3, or MAGE A3) has been found to be expressed in a variety of human tumour types of unrelated histological origin, including melanoma, non-small cell lung carcinoma (NSCLC), bladder cancer, head and neck cancers, squamous oesophageal cancer, and hepatocarcinoma. However, not all individuals having a certain tumor type (such as melanoma) will have a tumor that expresses MAGE-A3. Additionally, within a single tumor, some tumor cells may express a given cancer-testis antigen whereas other cells do not.

In general terms, a MAGE-A protein can be defined as containing a core sequence signature located towards the C-terminal end of the protein (for example with respect to the 309 amino acid MAGE-A1 protein, the core signature corresponds to amino acids 195-279). The consensus pattern of the core signature is set forth below (SEQ ID NO:1), wherein x represents any amino acid, lower case residues are conserved (conservative variants allowed) and upper case residues are perfectly conserved.

### MAGE-A Core Sequence Signature:

(SEQ ID NO: 1)
LixvL(2x)I(3x)g(2x)apEExiWexI(2x)m(3-4x)Gxe(3-4x)
gxp(2x)IIt(3x) VqexYLxYxqVPxsxP(2x)yeFLWGprA(2x)Et
(3x)ky

Conservative substitutions are well known and are generally set up as the default scoring matrices in sequence alignment computer programs. These programs include PAM250 (Dayhoft M. O. et al., (1978), “A model of evolutionary changes in proteins”, In “Atlas of Protein sequence and structure” 5(3) M. O. Dayhoft (ed.), 345-352), National Biomedical Research Foundation, Washington, and Blosum 62 (Steven Henikoft and Jorja G. Henikoft (1992), “Amino acid substitution matrices from protein blocks”), Proc. Natl. Acad. Sci. USA 89 (Biochemistry): 10915-10919.

As used herein, “adjuvant” means a compound or substance that, when administered to a subject in conjunction with a vaccine, immunotherapeutic, or other antigen- or immunogen-containing composition, increases or enhances the subject's immune response to the administered antigen or immunogen (as compared to the immune response that would be obtained in the absence of adjuvant). This is to be distinguished from “adjuvant therapy”, defined by the National Cancer Institute of the United States Institutes of Health in the context of cancer treatment as additional treatment given after the primary treatment, to lower the risk that the cancer will recur.

As used herein, a “susceptible cancer” is one that is positive for expression of MAGEA3 protein, assessed using any suitable method. See, e.g., Trefzer et al., Melanoma Research, 20 (eSupplement A):e34-e35 (June 2010).

Immunotherapeutics suitable for use in the invention are those capable of raising a MAGE-A3 specific immune response (a “MAGEA3 immunotherapeutic”). The immunotherapeutic will contain a MAGEA3 antigen comprising at least one epitope from a MAGEA3 gene product. Such an epitope may be present as a peptide antigen. Alternatively, larger protein fragments or full length MAGEA3 may be used. The MAGEA3 antigen must however, when suitably presented be capable of raising a MAGE-A3 specific immune response. The MAGEA3 protein, peptide or fragment may be joined to a fusion partner to provide a fusion protein.

The MAGE-A3 antigen and fusion partner may be chemically conjugated, or may be expressed as a recombinant fusion protein. In an embodiment in which the antigen and fusion partner are expressed as a recombinant fusion protein, this may allow increased levels to be produced in an expression system compared to non-fused antigen. Thus the fusion partner may assist in providing T helper epitopes (immunological fusion partner), including T helper epitopes recognised by humans, and/or assist in expressing the protein (expression enhancer) at higher yields than the native recombinant protein. In one embodiment, the fusion partner may be both an immunological fusion partner and expression enhancing partner.

In one embodiment of the invention, the immunological fusion partner is derived from protein D, a surface protein of the gram-negative bacterium, Haemophilus influenza B (WO91/18926) or a derivative thereof. Protein D is synthesized as a precursor with an 18-residue-long signal sequence; the cysteine residue at amino acid position 19 of the unprocessed Protein D becomes the amino terminus after cleavage of the signal sequence. (Janson et al., Infection & Immunology 60(4):1336-42 (1992)). In one embodiment, the protein D or fragment thereof may be lipidated.

In one embodiment, the protein D derivative comprises approximately the first ⅓ of the processed protein, in particular approximately the first N-terminal 100-120 amino acids such as the first 109 to 112 amino acids, more specifically the first 109 amino acids (or 108 amino acids thereof). In one embodiment, the protein D derivative may comprise amino acids 20 to 127 of processed protein D. The first 109 residues of the Lipoprotein D fusion partner may provide additional exogenous T-cell epitopes and increase expression level in Escherichia coli (thus acting as both an immunological fusion partner and as an expression enhancer). In one embodiment, the above portions of Protein D additionally include the 18 amino acid signal sequence. Thus the immunotherapeutic fusion protein may comprise the N-terminal portion of protein D as described herein fused to the N-terminus of the MAGEA3 antigen, or an immunogenic fragment thereof. The fusion of the protein D and the N-terminus of the MAGEA3 antigen may occur such that the MAGEA3 antigen replaces the excised C-terminal-fragment of protein D and the N-terminus of protein D becomes the N-terminus of the fusion protein.

Other fusion partners may be used in therapeutic fusion proteins as described herein, in place of or in addition to protein D. One such fusion partner is the non-structural protein from influenzae virus, NS1 (hemagglutinin); typically the N terminal 81 amino acids are utilised, although different fragments may be used provided they include T-helper epitopes.

In another embodiment the immunological fusion partner is the protein known as LytA. LytA is derived from Streptococcus pneumoniae which synthesise an N-acetyl-L-alanine amidase, amidase LytA, (coded by the LytA gene {Garcia et al., Gene, 43 (1986) page 265-272}) an autolysin that specifically degrades certain bonds in the peptidoglycan backbone. The C-terminal domain of the LytA protein is responsible for the affinity to the choline or to some choline analogues such as DEAE. This property has been exploited for the development of E. coli C-LytA expressing plasmids useful for expression of fusion proteins. Purification of hybrid proteins containing the C-LytA fragment at its amino terminus has been described (Ortega, et al., Biotechnology: 10, (1992) page 795-798). In one embodiment, the C terminal portion of the molecule may be used. The embodiment may utilise the repeat portion of the LytA molecule found in the C terminal end starting at residue 178. In one embodiment, the LytA portion may incorporate residues 188-305.

Immunotherapeutic fusion proteins for use in the present invention may include an affinity tag, such as for example, a histidine tail comprising between 5 to 9, such as 6 to 7, histidine residues. These residues may, for example, be on the terminal portion of protein D (such as the N-terminal of protein D) and/or may be fused to the terminal portion of the MAGEA3 antigen. Generally however the histidine tail with be located on terminal portion of the antigen, such as the C-terminal end of the antigen. Histidine tails are advantageous in aiding purification.

MAGE-A3 and fusion proteins thereof useful in the present invention are described in WO99/40188; EP1053325; EP1659178. The use of a MAGE-A3 immunotherapeutic in combination with other anti-cancer treatments such as surgery, chemotherapy and/or radiotherapy is described in WO2008/084040. In one embodiment of the present invention the immunotherapeutic comprises a fusion protein of Protein D and MAGE-A3, where the fusion protein comprises approximately or exactly the first 127 amino acids of unprocessed protein D (with or without replacement of amino acids K-2 and L-3 of protein D by amino acids D-2 and P-3, see below; this numbering is for the amino acid sequence of protein D including the 18 amino acid signal sequence). In one embodiment, the Protein D-MAGE-A3 fusion protein does not include the 18 amino acid signal sequence of protein D. The fusion protein may include one or two linker amino acids before the protein D sequence and/or the MAGE-A3 sequence. The fusion protein may further comprise an optional histidine tail, for example seven histidine residues, and may further comprise one or two linker amino acids between the MAGE-3 sequence and the His tail. One such Protein D-MAGE-A3 fusion protein has the following sequence (SEQ ID NO:2); this construct is referred to herein as recMAGEA3:

| | |
|---|---|
| MDPKTLALSL LAAGVLAGCS SHSSNMANTQ MKSDKIIIAH | 40 |
| RGASGYLPEH TLESKALAFA QQADYLEQDL AMTKDGRLVV | 80 |
| IHDHFLDGLT DVAKKFPHRH RKDGRYYVID FTLKEIQSLE | 120 |
| MTENFETMDL EQRSQHCKPE EGLEARGEAL GLVGAQAPAT | 160 |
| EEQEAASSSS TLVEVTLGEV PAAESPDPPQ SPQGASSLPT | 200 |
| TMNYPLWSQS YEDSSNQEEE GPSTFPDLES EFQAALSRKV | 240 |
| AELVHFLLLK YRAREPVTKA EMLGSVVGNW QYFFPVIFSK | 280 |
| ASSSLQLVFG IELMEVDPIG HLYIFATCLG LSYDGLLGDN | 320 |
| QIMPKAGLLI IVLAIIAREG DCAPEEKIWE ELSVLEVFEG | 360 |
| REDSILGDPK KLLTQHFVQE NYLEYRQVPG SDPACYEFLW | 400 |
| GPRALVETSY VKVLHHMVKI SGGPHISYPP LHEWVLREGE | 440 |
| EGGHHHHHHH | 450 |

In SEQ ID NO:2, the first 127 amino acids are Protein D, including the initial 18 amino acid signal sequence and having Asp-Pro substituted at residues 2-3; at residues 128-129 Met-Asp is between the Protein D sequence and the MAGE-A3 sequence; residues 130-441 are 312 amino acids of MAGE-A3; Gly-Gly at residues 442-443 are placed between the MAGE-A3 sequence and the 7 histidine tail. See, e.g., WO 99/40188; EP1053325; and EP1659178. Alternatively, amino acids 128-441of recMAGEA3 could be described as full-length (314 amino acids) MAGE-A3 having Asp substituted at the second amino acid position.

In another aspect of the invention, there is provided a a method of treating a susceptible cancer in a human in need thereof, said method comprising administering a combination comprising

(i) recMAGEA3, and

### (ii) Compound A.

In another aspect of the invention, there is provided a a method of treating a susceptible cancer in a human in need thereof, said method comprising administering a combination comprising

(i) recMAGEA3, and

### (ii) Compound B.

In another aspect of the invention, there is provided a a method of treating a susceptible cancer in a human in need thereof, said method comprising administering a combination comprising

(i) recMAGEA3,

### (ii) Compound A, and

(iii) Compound B.

In another aspect of the invention, there is provided a a method of treating a susceptible melanoma in a human in need thereof, said method comprising administering a combination comprising

(i) recMAGEA3, and

### (ii) Compound A.

In another aspect of the invention, there is provided a a method of treating a susceptible melanoma in a human in need thereof, said method comprising administering a combination comprising

(i) recMAGEA3, and

### (ii) Compound B.

In another aspect of the invention, there is provided a a method of treating a susceptible melanoma in a human in need thereof, said method comprising administering a combination comprising

(i) recMAGEA3,

### (ii) Compound A, and

(iii) Compound B.

In another aspect of the invention, there is provided a method of treating a susceptible cancer in a human in need thereof, said method comprising administering a combination comprising

(i) recMAGEA3, and
(ii) N-{3-[5-(2-Amino-4-pyrimidinyl)-2-(1,1-dimethylethyl)-1,3-thiazol-4-yl]-2-fluorophenyl}-2,6-difluorobenzenesulfonamide methanesulfonate.

In another aspect of the invention, there is provided a method of treating a susceptible cancer in a human in need thereof, said method comprising administering a combination comprising

(i) recMAGEA3, and
(ii) N-{3-[3-cyclopropyl-5-(2-fluoro-4-iodo-phenylamino)6,8-dimethy; -2,4,7-trioxo-3,4,6,7-tetrahydro-2H-pyrido[4,3-d]pyrimidin-1-yl]phenyl}acetamide dimethyl sulfoxide.

In another aspect of the invention, there is provided a method of treating a susceptible cancer in a human in need thereof, said method comprising administering a combination comprising

(i) recMAGEA3,
(ii) N-{3-[5-(2-Amino-4-pyrimidinyl)-2-(1,1-dimethylethyl)-1,3-thiazol-4-yl]-2-fluorophenyl}-2,6-difluorobenzenesulfonamide methanesulfonate, and
(iii) N-{3-[3-cyclopropyl-5-(2-fluoro-4-iodo-phenylamino)6,8-dimethy; -2,4,7-trioxo-3,4,6,7-tetrahydro-2H-pyrido[4,3-d]pyrimidin-1-yl]phenyl}acetamide dimethyl sulfoxide.

In another aspect of the present invention, there is provided a method of treating a susceptible cancer in a human in need thereof, said method comprising administering a combination comprising

(i) recMAGEA3, and
(ii) Compound A, together with a pharmaceutically acceptable diluent or carrier.

In another aspect of the present invention, there is provided a method of treating a susceptible cancer in a human in need thereof, said method comprising administering a combination comprising

(i) recMAGEA3, and
(ii) Compound B, together with a pharmaceutically acceptable diluent or carrier.

In another aspect of the present invention, there is provided a method of treating a susceptible cancer in a human in need thereof, said method comprising administering a combination comprising

(i) recMAGEA3,
(ii) Compound A, together with a pharmaceutically acceptable diluent or carrier, and
(iii) Compound B, together with a pharmaceutically acceptable diluent or carrier.

In a another aspect there is provided the use of a combination comprising

(i) recMAGEA3 and
(ii) Compound A; for the treatment of a susceptible cancer in a human.

In a another aspect there is provided the use of a combination comprising

(i) recMAGEA3 and
(ii) Compound B; for the treatment of a susceptible cancer in a human.

In a another aspect there is provided the use of a combination comprising

(i) recMAGEA3

### (ii) Compound A, and

(iii) Compound B, for the treatment of a susceptible cancer in a human.

In one embodiment of the present invention, the MAGE-A3 protein may comprise a derivatised free thiol. Such antigens and methods of producing them have been described in WO99/40188. In particular carboxyamidated or carboxymethylated derivatives may be used.

See also Kruit et al., Immunization with recombinant MAGE-A3 protein combined with adjuvant systems AS15 or AS02B in patients with unresectable and progressive metastatic cutaneous melanoma: A randomized open-label phase II study of the EORTC Melanoma Group (16032-18031), ASCO Meeting Abstracts 2008 J. Clinical Oncology 26(May 20 Suppl; Abstract 9065) (2008).

Alternatively, vectors comprising DNA encoding the immunotherapeutic protein may be administered. An immune response may generated against the vector carrying the encoding DNA and thus the general immune response may be boosted (i.e., the vector is itself acting as an adjuvant). The immunotherapy may, for example be administered as a prime boost regime.

Thus, the present invention may be used to treat human subjects having MAGE-A3 expressing cancers, such as melanoma, breast, bladder, non-small cell lung cancer (NSCLC), colon, esophageal and head and neck squamous cell carcinoma.

### BRAF Inhibitor

As used herein, the BRaf inhibitor N-{3-[5-(2-Amino-4-pyrimidinyl)-2-(1,1-dimethylethyl)-1,3-thiazol-4-yl]-2-fluorophenyl}-2,6-difluorobenzenesulfonamide or pharmaceutically acceptable salt thereof, is represented by a compound formula (I):

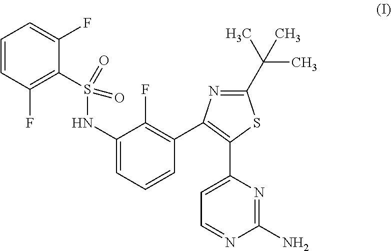 [SMILES: CC(C)(C)c1nc(-c2cccc(NS(=O)(=O)c3c(F)cccc3F)c2F)c(-c2ccnc(N)n2)s1]

or a pharmaceutically acceptable salt thereof, For convenience, the group of possible compound and salts is collectively referred to as Compound A, meaning that reference to Compound A will refer to any of the compound or pharmaceutically acceptable salt thereof in the alternative.

Compound A is disclosed and claimed, along with pharmaceutically acceptable salts thereof, as being useful as an inhibitor of BRaf activity, particularly in the treatment of cancer, in PCT patent application PCT/US09/42682. Compound A is embodied by Examples 58a through 58e of the application. The PCT application was published on 12 Nov. 2009 as publication WO2009/137391, and is hereby incorporated by reference.

Typically, the salts of the present invention are pharmaceutically acceptable salts. Salts encompassed within the term “pharmaceutically acceptable salts” refer to non-toxic salts of the compounds of this invention. Salts of the compounds of the present invention may comprise acid addition salts derived from a nitrogen on a substituent in a compound of the present invention. Representative salts include the following salts: acetate, benzenesulfonate, benzoate, bicarbonate, bisulfate, bitartrate, borate, bromide, calcium edetate, camsylate, carbonate, chloride, clavulanate, citrate, dihydrochloride, edetate, edisylate, estolate, esylate, fumarate, gluceptate, gluconate, glutamate, glycollylarsanilate, hexylresorcinate, hydrabamine, hydrobromide, hydrochloride, hydroxynaphthoate, iodide, isethionate, lactate, lactobionate, laurate, malate, maleate, mandelate, mesylate, methylbromide, methylnitrate, methylsulfate, monopotassium maleate, mucate, napsylate, nitrate, N-methylglucamine, oxalate, pamoate (embonate), palmitate, pantothenate, phosphate/diphosphate, polygalacturonate, potassium, salicylate, sodium, stearate, subacetate, succinate, tannate, tartrate, teoclate, tosylate, triethiodide, trimethylammonium and valerate. Other salts, which are not pharmaceutically acceptable, may be useful in the preparation of compounds of this invention and these form a further aspect of the invention. Salts may be readily prepared by a person skilled in the art.

Compound A may be presented as a solvate. As used herein, the term “solvate” refers to a complex of variable stoichiometry formed by a solute (in this invention, compound of formula (I) or a salt thereof, and a solvent. Such solvents for the purpose of the invention may not interfere with the biological activity of the solute. Examples of suitable solvents include, but are not limited to, water, methanol, dimethylsulforide. ethanol and acetic acid. In one embodiment, the solvent used is a pharmaceutically acceptable solvent.

Pharmaceutical compositions may be presented in unit dose forms containing a predetermined amount of active ingredient per unit dose. As is known to those skilled in the art, the amount of active ingredient per dose will depend on the condition being treated, the route of administration and the age, weight and condition of the patient. Preferred unit dosage compositions are those containing a daily dose or sub-dose, or an appropriate fraction thereof, of an active ingredient. Furthermore, such pharmaceutical compositions may be prepared by any of the methods well known in the pharmacy art. More particular methods of administration are described in PCT publication WO2009/137391.

Suitably, the amount of Compound A (based on weight of unsalted/unsolvated amount) administered as part of the combination according to the present invention will be an amount selected from about 10 mg to about 600 mg. Suitably, the amount will be selected from about 30 mg to about 300 mg; suitably, the amount will be selected from about 30 mg to about 280 mg; suitably, the amount will be selected from about 40 mg to about 260 mg; suitably, the amount will be selected from about 60 mg to about 240 mg; suitably, the amount will be selected from about 80 mg to about 220 mg; suitably, the amount will be selected from about 90 mg to about 210 mg; suitably, the amount will be selected from about 100 mg to about 200 mg, suitably, the amount will be selected from about 110 mg to about 190 mg, suitably, the amount will be selected from about 120 mg to about 180 mg, suitably, the amount will be selected from about 130 mg to about 170 mg, suitably, the amount will be selected from about 140 mg to about 160 mg, suitably, the amount will be 150 mg. Accordingly, the amount of Compound A administered as part of the combination according to the present invention will be an amount selected from about 10 mg to about 300 mg. For example, the amount of Compound A administered as part of the combination according to the present invention is suitably selected from 10 mg, 20 mg, 30 mg, 40 mg, 50 mg, 60 mg, 70 mg, 80 mg, 85 mg, 90 mg, 95 mg, 100 mg, 105 mg, 110 mg, 115 mg, 120 mg, 125 mg, 130 mg, 135 mg, 140 mg, 145 mg, 150 mg, 155 mg, 160 mg, 165 mg, 170 mg, 175 mg, 180 mg, 185 mg, 190 mg, 195 mg, 200 mg, 205 mg, 210 mg, 215 mg, 220 mg, 225 mg, 230 mg, 235 mg, 240 mg, 245 mg, 250 mg, 255 mg, 260 mg, 265 mg, 270 mg, 275 mg, 280 mg, 285 mg, 290 mg, 295 mg and 300 mg. Suitably, the selected amount of Compound A is administered from 1 to 4 times a day. Suitably, the selected amount of Compound A is administered twice a day. Suitably, Compound A is administered at an amount of 150 mg twice a day. Suitably, the selected amount of Compound A is administered once a day.

### MEK Inhibitor

As used herein, the MEK inhibitor N-{3-[3-cyclopropyl-5-(2-fluoro-4-iodo-phenylamino)6,8-dimethy; -2,4,7-trioxo-3,4,6,7-tetrahydro-2H-pyrido[4,3-d]pyrimidin-1-yl]phenyl}acetamide or pharmaceutically acceptable salt or solvate thereof, is represented by a compound of structure (I):

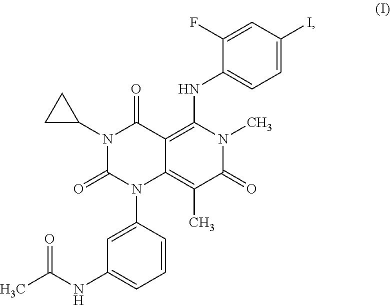 [SMILES: CC(=O)Nc1cccc(-n2c(=O)n(C3CC3)c(=O)c3c(Nc4ccc(I)cc4F)n(C)c(=O)c(C)c32)c1]

or a pharmaceutically acceptable salt or solvate thereof. For convenience, the group of possible compound and/or salts and/or solvates is collectively referred to as Compound B, meaning that reference to Compound B will refer to any of the compound and/or pharmaceutically acceptable salts and/or solvates thereof in the alternative.

Compound B is disclosed and claimed, along with pharmaceutically acceptable salts and solvates thereof, as being useful as an inhibitor of MEK activity, particularly in treatment of cancer, in International Application No. PCT/JP2005/011082, having an International filing date of Jun. 10, 2005; International Publication Number WO 2005/121142 and an International Publication date of Dec. 22, 2005, the entire disclosure of which is hereby incorporated by reference, Compound B is the compound of Example 4-1. Compound B can be prepared as described in International Application No. PCT/JP2005/011082. Compound B can be prepared as described in United States Patent Publication No. US 2006/0014768, Published Jan. 19, 2006, the entire disclosure of which is hereby incorporated by reference.

Suitably, Compound B is in the form of a dimethyl sulfoxide solvate. Suitably, Compound B is in the form of a sodium salt. Suitably, Compound B is in the form of a solvate selected from: hydrate, acetic acid, ethanol, nitromethane, chlorobenzene, 1-pentanci, isopropyl alcohol, ethylene glycol and 3-methyl-1-butanol. These solvates and salt forms can be prepared by one of skill in the art from the description in International Application No. PCT/JP2005/011082 or United States Patent Publication No. US 2006/0014768.

Typically, the salts of the present invention are pharmaceutically acceptable salts. Salts encompassed within the term “pharmaceutically acceptable salts” refer to non-toxic salts of the compounds of this invention. Salts of the compounds of the present invention may comprise acid addition salts derived from a nitrogen on a substituent in a compound of the present invention. Other salts, which are not pharmaceutically acceptable, may be useful in the preparation of compounds of this invention and these form a further aspect of the invention. Salts may be readily prepared by a person skilled in the art.

Compound B may be presented as a solvate. As used herein, the term “solvate” refers to a complex of variable stoichiometry formed by a solute (in this invention, compound of structure (I) or a salt thereof), and a solvent. Such solvents for the purpose of the invention may not interfere with the biological activity of the solute. Examples of suitable solvents include, but are not limited to, water, methanol, dimethylsulfoxide. ethanol and acetic acid. In one embodiment, the solvent used is a pharmaceutically acceptable solvent.

Pharmaceutical compositions may be presented in unit dose forms containing a predetermined amount of active ingredient per unit dose. As is known to those skilled in the art, the amount of active ingredient per dose will depend on the condition being treated, the route of administration and the age, weight and condition of the patient. Preferred unit dosage compositions are those containing a daily dose or sub-dose, or an appropriate fraction thereof, of an active ingredient. Furthermore, such pharmaceutical compositions may be prepared by any of the methods well known in the pharmacy art. More particular methods of administration are described in United States Patent Publication No. US 2006/0014768.

Suitably, the amount of Compound B (based on weight of unsalted/unsolvated amount) administered as part of the combination according to the present invention will be an amount selected from about 0.125 mg to about 10 mg; suitably, the amount will be selected from about 0.25 mg to about 9 mg; suitably, the amount will be selected from about 0.25 mg to about 8 mg; suitably, the amount will be selected from about 0.5 mg to about 8 mg; suitably, the amount will be selected from about 0.5 mg to about 7 mg; suitably, the amount will be selected from about 1 mg to about 7 mg; suitably, the amount will be about 5 mg. Accordingly, the amount of Compound B administered as part of the combination according to the present invention will be an amount selected from about 0.125 mg to about 10 mg. For example, the amount of Compound B administered as part of the combination according to the present invention can be 0.125 mg, 0.25 mg, 0.5 mg, 0.75 mg, 1 mg, 1.5 mg, 2 mg, 2.5 mg, 3 mg, 3.5 mg, 4 mg, 4.5 mg, 5 mg, 5.5 mg, 6 mg, 6.5 mg, 7 mg, 7.5 mg, 8 mg, 8.5 mg, 9 mg, 9.5 mg, 10 mg. Suitably, the selected amount of Compound B is administered from 1 to 4 times a day. Suitably, the selected amount of Compound B is administered twice a day. Suitably, the selected amount of Compound B is administered once a day.

### Method of Treatment

The combinations of the invention, are believed to have utility in disorders wherein the inhibition of B-Raf and/or MEK activity and the raising of a specific immune response to MAGEA3 is beneficial. The present invention thus also provides a combination of the invention, for use in therapy, particularly in the treatment of disorders wherein the inhibition of B-Raf and/or MEK activity and the raising of a specific immune response to MAGEA3 is beneficial, particularly cancer.

A further aspect of the invention provides a method of treatment of a disorder wherein to inhibition of B-Raf and/or MEK activity and the raising of a specific immune response to MAGEA3 is beneficial, comprising administering a combination of the invention.

A further aspect of the present invention provides the use of a combination of the invention in the manufacture of a medicament for the treatment of a disorder wherein the inhibition of B-Raf and/or MEK activity and the raising of a specific immune response to MAGEA3 is beneficial.

As used herein, “concurrent or concomitant administration” refers to administration of two (or more) therapies such that the therapeutic moieties are introduced into the body at the same time, or close enough in time that the first administered therapy is still in the subject's system (has not been metabolised, excreted or the like) at the time subsequent therapy(ies) are administered. Administration may be by different routes. As used herein the terms concurrently and concomitantly are substitutable.

As used herein, “response to treatment” means a response to anti-cancer treatment may be measured in any way as is known and accepted in the art, including by following the response of the tumor (complete regression of the tumor(s) (complete response), reduction in size or volume of the tumor(s) (partial response); no apparent growth or progression of tumor(s) (stable disease), or mixed response (regression or stabilization of some tumors but not others)). Alternatively, the effect of anti-cancer treatment may be assessed by following the patient, e.g., by measuring and comparing survival time, or time to disease progression (disease-free survival). Any assessment of response may be compared to individuals who did not receive the treatment, or to individuals who received an alternative treatment.

Regarding the MAGE-A3 immunotherapeutic, response to treatment may be predicted by detection of gene signatures within the human to be treated. WO2010/029174; WO2009/068621; and WO2007/140958 describe methods of classifying a subject as a responder or non-responder to treatment with the immunotherapeutic, by detecting the expression levels or differential expression of certain genes (including many immune-related genes) in the tumor microenvironment, prior to treatment. Subjects classified as responders are more likely to respond to treatment with an immune based therapy such as a MAGEA3 immunotherapy. In one embodiment, such methods are useful when considering treating a subject having melanoma with recMAGEA3. See e.g., Louahed et al., Expression of defined genes identified by pretreatment tumor profiling: Association with clinical responses to the GSK MAGE-A3 immunotherapeutic in metastatic melanoma patients (EORTC 16032-18031), ASCO Meeting Abstracts 2008, J. Clinical Oncology 26(May 20 Suppl; Abstract 9045) (2008).

The combination of the invention is suitable for use in treatment of a cancer such that inhibition of B-Raf and/or MEK activity and the raising of a specific immune response to MAGEA3 has a beneficial effect. Examples of cancers that are suitable for treatment with combination of the invention include, but are limited to, both primary and metastatic forms of head and neck, breast, lung, non-small cell lung cancer (NSCLC), colon, ovary, and prostate cancers. Suitably the cancer is selected from: brain (gliomas), glioblastomas, astrocytomas, glioblastoma multiforme, Bannayan-Zonana syndrome, Cowden disease, Lhermitte-Duclos disease, breast, inflammatory breast cancer, Wilm's tumor, Ewing's sarcoma, Rhabdomyosarcoma, ependymoma, medulloblastoma, colon, head and neck, kidney, lung, liver, melanoma, ovarian, pancreatic, prostate, sarcoma, osteosarcoma, giant cell tumor of bone, thyroid cancer, lymphoblastic T cell leukemia, Chronic myelogenous leukemia, Chronic lymphocytic leukemia, Hairy-cell leukemia, acute lymphoblastic leukemia, acute myelogenous leukemia, AML, Chronic neutrophilic leukemia, Acute lymphoblastic T cell leukemia, plasmacytoma, Immunoblastic large cell leukemia, Mantle cell leukemia, Multiple myeloma Megakaryoblastic leukemia, multiple myeloma, acute megakaryocytic leukemia, promyelocytic leukemia, Erythroleukemia, malignant lymphoma, hodgkins lymphoma, non-hodgkins lymphoma, lymphoblastic T cell lymphoma, Burkitt's lymphoma, follicular lymphoma, neuroblastoma, bladder cancer, urothelial cancer, lung cancer, vulval cancer, cervical cancer, endometrial cancer, renal cancer, mesothelioma, esophageal cancer, salivary gland cancer, hepatocellular cancer, gastric cancer, nasopharangeal cancer, buccal cancer, cancer of the mouth, GIST (gastrointestinal stromal tumor) and testicular cancer.

Additionally, examples of a cancer to be treated include Barret's adenocarcinoma; billiary tract carcinomas; breast cancer; cervical cancer; cholangiocarcinoma; central nervous system tumors including primary CNS tumors such as glioblastomas, astrocytomas (e.g., glioblastoma multiforme) and ependymomas, and secondary CNS tumors (i.e., metastases to the central nervous system of tumors originating outside of the central nervous system); colorectal cancer including large intestinal colon carcinoma; gastric cancer; carcinoma of the head and neck including squamous cell carcinoma of the head and neck; hematologic cancers including leukemias and lymphomas such as acute lymphoblastic leukemia, acute myelogenous leukemia (AML), myelodysplastic syndromes, chronic myelogenous leukemia, Hodgkin's lymphoma, non-Hodgkin's lymphoma, megakaryoblastic leukemia, multiple myeloma and erythroleukemia; hepatocellular carcinoma; lung cancer including small cell lung cancer and non-small cell lung cancer; ovarian cancer; endometrial cancer; pancreatic cancer; pituitary adenoma; prostate cancer; renal cancer; sarcoma; skin cancers including melanomas; and thyroid cancers.

Suitably, the present invention relates to a method for treating or lessening the severity of a cancer selected from BRAF V600-mutant, MAGE-A3 positive tumors, such as BRAF V600-mutant MAGE-A3 positive brain (gliomas), glioblastomas, Bannayan-Zonana syndrome, Cowden disease, Lhermitte-Duclos disease, breast, colon, head and neck, kidney, lung, liver, melanoma, ovarian, pancreatic, prostate, sarcoma and thyroid.

Additionally, the present invention relates to a method for treating or lessening the severity of a cancer selected from BRAF V600E-mutant, MAGE-A3 positive tumors, such as BRAF V600E-mutant MAGE-A3 positive brain (gliomas), glioblastomas, Bannayan-Zonana syndrome, Cowden disease, Lhermitte-Duclos disease, breast, colon, head and neck, kidney, lung, liver, melanoma, ovarian, pancreatic, prostate, sarcoma and thyroid.

Suitably, the present invention relates to a method for treating or lessening the severity of melanoma, particularly BRAF V600-mutant MAGE-A3 positive melanoma, more particularly BRAF V600E-mutant MAGE-A3 positive melanoma.

Unless otherwise defined, in all dosing protocols described herein, the regimen of combined B-Raf and/or MEK inhibitor compound and MAGEA3 immunotherapeutic does not have to commence at the start of treatment and terminate with the end of treatment. It is only required that at some point during treatment both the B-Raf and/or MEK inhibitor and the MAGEA3 immunotherapeutic be administered on the same days. B-Raf inhibitors may be administered in daily doses, whereas administration of a MAGEA3 immunotherapeutic may occur at intervals of several weeks followed by additional administrations every several months (see, e.g., WO2007/137986).

As used herein the term “neoplasm” refers to an abnormal growth of cells or tissue and is understood to include benign, i.e., non-cancerous growths, and malignant, i.e., cancerous growths. The term “neoplastic” means of or related to a neoplasm.

As used herein the term “agent” is understood to mean a substance that produces a desired effect in a tissue, system, animal, mammal, human, or other subject. Accordingly, the term “anti-neoplastic agent” is understood to mean a substance producing an anti-neoplastic effect in a tissue, system, animal, mammal, human, or other subject. It is also to be understood that an “agent” may be a single compound, single antigen, or a combination or composition of two or more compounds or antigens.

By the term “treating” and derivatives thereof as used herein, is meant therapeutic therapy. In reference to a particular condition, treating means: (1) to ameliorate the condition or one or more of the biological manifestations of the condition, (2) to interfere with (a) one or more points in the biological cascade that leads to or is responsible for the condition or (b) one or more of the biological manifestations of the condition (3) to alleviate one or more of the symptoms, effects or side effects associated with the condition or one or more of the symptoms, effects or side effects associated with the condition or treatment thereof, or (4) to slow the progression of the condition or one or more of the biological manifestations of the condition.

As used herein, “prevention” is understood to refer to the prophylactic administration of a drug to substantially diminish the likelihood or severity of a condition or biological manifestation thereof, or to delay the onset of such condition or biological manifestation thereof. The skilled artisan will appreciate that “prevention” is not an absolute term. Prophylactic therapy is appropriate, for example, when a subject is considered at high risk for developing cancer, such as when a subject has a strong family history of cancer or when a subject has been exposed to a carcinogen.

As used herein, the term “effective amount” means that amount of a drug or pharmaceutical agent that will elicit the biological or medical response of a tissue, system, animal or human that is being sought, for instance, by a researcher or clinician. Furthermore, the term “therapeutically effective amount” means any amount which, as compared to a corresponding subject who has not received such amount, results in improved treatment, healing, prevention, or amelioration of a disease, disorder, or side effect, or a decrease in the rate of advancement of a disease or disorder. The term also includes within its scope amounts effective to enhance normal physiological function.

Compound A and/or Compound B, and the MAGE-A3 immunotherapeutic may be employed in either concurrent or concomitant administration. Thus in one embodiment, one or more doses of Compound A are administered simultaneously or separately with one or more doses of MAGE-A3 fusion protein.

The term “loading dose” as used herein will be understood to mean a single dose or short duration regimen of Compound A, Compound B, and/or the MAGE-A3 immunotherapeutic having a dosage higher than the maintenance dose administered to the subject to, for example, rapidly increase the blood concentration level of the drug. The term “maintenance dose” as used herein will be understood to mean a dose that is serially administered (for example; at least twice), and which is intended to either slowly raise blood concentration levels of the compound to a therapeutically effective level, or to maintain such a therapeutically effective level. The maintenance dose is generally administered once per day and the daily dose of the maintenance dose is lower than the total daily dose of the loading dose.

In one embodiment the mammal in the methods and uses of the present invention is a human.

Suitably, the present invention relates to a method of treating or lessening the severity of a cancer that is either wild type or mutant for each of Raf, Ras, MEK, and PI3K/Pten. This includes but is not limited to patients having cancers that are mutant for RAF, wild type for RAS, wild type for MEK, and wild type for PI3K/PTEN; mutant for RAF, mutant for RAS, wild type for MEK, and wild type for PI3K/PTEN; mutant for RAF, mutant for RAS, mutant for MEK, and wild type for PI3K/PTEN; and mutant for RAF, wild type for RAS, mutant for MEK, and wild type PI3K/PTEN.

The term “wild type” as is understood in the art refers to a polypeptide or polynucleotide sequence that occurs in a native population without genetic modification. As is also understood in the art, a “mutant” includes a polypeptide or polynucleotide sequence having at least one modification to an amino acid or nucleic acid compared to the corresponding amino acid or nucleic acid found in a wild type polypeptide or polynucleotide, respectively. Included in the term mutant is Single Nucleotide Polymorphism (SNP) where a single base pair distinction exists in the sequence of a nucleic acid strand compared to the most prevalently found (wild type) nucleic acid strand.

Cancers that are either wild type or mutant for Raf, Ras, MEK, or mutant for PI3K/Pten are identified by known methods. For example, wild type or mutant tumor cells can be identified by DNA amplification and sequencing techniques, DNA and RNA detection techniques, including, but not limited to Northern and Southern blot, respectively, and/or various biochip and array technologies. Wild type and mutant polypeptides can be detected by a variety of techniques including, but not limited to immunodiagnostic techniques such as ELISA, Western blot or immunocyto chemistry. Suitably, Pyrophosphorolysis-activated polymerization (PAP) and/or PCR methods may be used. Liu, Q et al; Human Mutation 23:426-436 (2004).

As indicated, therapeutically effective amounts of Compound A is discussed above. The therapeutically effective amount of the further therapeutic agents of the present invention will depend upon a number of factors including, for example, the age and weight of the mammal, the precise condition requiring treatment, the severity of the condition, the nature of the formulation, and the route of administration. Ultimately, the therapeutically effective amount will be at the discretion of the attendant physician or veterinarian. The relative timings of administration will be selected in order to achieve the desired combined therapeutic effect.

### Combinations

In one embodiment, the invented method of treatment includes administration of the disclosed BRaf inhibitor and/or MEK inhibitor and MAGE-A3 immunotherapeutic, and at least one additional anti-neoplastic agent.

Typically, any anti-neoplastic agent that has activity versus a susceptible tumor being treated may be co-administered in the treatment of cancer in the present invention. Examples of such agents can be found in Cancer Principles and Practice of Oncology by V. T. Devita and S. Hellman (editors), 6^{th} edition (Feb. 15, 2001), Lippincott Williams & Wilkins Publishers. Typical anti-neoplastic agents useful for combination with the BRaf and MEK inhibitors discussed above include, but are not limited to, anti-microtubule agents such as diterpenoids and vinca alkaloids; platinum coordination complexes; alkylating agents such as nitrogen mustards, oxazaphosphorines, alkylsulfonates, nitrosoureas, and triazenes; antibiotic agents such as anthracyclins, actinomycins and bleomycins; topoisomerase II inhibitors such as epipodophyllotoxins; antimetabolites such as purine and pyrimidine analogues and anti-folate compounds; topoisomerase I inhibitors such as camptothecins; hormones and hormonal analogues; signal transduction pathway inhibitors; receptor tyrosine kinase inhibitors; serine-threonine kinase inhibitors; non-receptor tyrosine kinase inhibitors; angiogenesis inhibitors, immunotherapeutic agents; proapoptotic agents; and cell cycle signalling inhibitors.

Anti-microtubule or anti-mitotic agents, such as diterpenoids and vinca alkaloids (such as vinblastine, vincristine, and vinorelbine); diterpenoids, such as paclitaxel (TAXOL®) and its analog docetaxel; platinum coordination complexes, such as cisplatin and carboplatin; alkylating agents, such as cyclophosphamide, melphalan, and chlorambucil; alkyl sulfonates such as busulfan; nitrosoureas such as carmustine; and triazenes such as dacarbazine.

Additional anti-neoplastic agents that may be used in combination with the invention include antibiotic anti-neoplastics, such as actinomycins such as dactinomycin, anthracyclins such as daunorubicin and doxorubicin; and bleomycins; topoisomerase II inhibitors, such as epipodophyllotoxins, such as etoposide and teniposide.

Additional anti-neoplastic agents that may be used in combination with the invention include antimetabolite neoplastic agents, such as fluorouracil (5-fluoro-2,4-(1H,3H) pyrimidinedione, 5-fluoro deoxyuridine (floxuridine) and 5-fluorodeoxyuridine monophosphate), methotrexate, cytarabine, mecaptopurine (PURINETHOL®), thioguanine (TABLOID®), and gemcitabine (GEMZAR®).

Additional anti-neoplastic agents that may be used in combination with the invention include camptothecins, including, camptothecin and camptothecin derivatives available or under development as Topoisomerase I inhibitors, such as irinotecan, topotecan, and the various optical forms of 7-(4-methylpiperazino-methylene)-10,11-ethylenedioxy-20-camptothecin; Irinotecan HCl (CAMPTOSAR®); Irinotecan; and Topotecan HCl (HYCAMTIN®).

Further anti-neoplastic agents that may be used in combination with the invention include rituximab (RITUXAN® and MABTHERA®); ofatumumab (ARZERRA®); mTOR inhibitors include but are not limited to rapamycin and rapalogs, RAD001 or everolimus (Afinitor), CCI-779 or temsirolimus, AP23573, AZD8055, WYE-354, WYE-600, WYE-687 and Pp121; bexarotene (Targretin®); and sorafenib (Nexavar®).

The invented combination may be used in combination with hormones useful in treating cancer. Examples of hormones and hormonal analogues useful in cancer treatment include, but are not limited to, adrenocorticosteroids such as prednisone and prednisolone; aminoglutethimide and other aromatase inhibitors such as anastrozole, letrazole, vorazole, and exemestane; progestrins such as megestrol acetate; estrogens, androgens, and anti-androgens such as flutamide, nilutamide, bicalutamide, cyproterone acetate and 5α-reductases such as finasteride and dutasteride; anti-estrogens such as tamoxifen, toremifene, raloxifene, droloxifene, iodoxyfene, as well as selective estrogen receptor modulators (SERMS) such those described in U.S. Pat. Nos. 5,681,835, 5,877,219, and 6,207,716; and gonadotropin-releasing hormone (GnRH) and analogues thereof which stimulate the release of leutinizing hormone (LH) and/or follicle stimulating hormone (FSH) for the treatment prostatic carcinoma, for instance, LHRH agonists and antagagonists such as goserelin acetate and luprolide.

The invented combination may be used in further combination with signal transduction pathway inhibitors, such as inhibitors of receptor tyrosine kinases, non-receptor tyrosine kinases, SH2/SH3domain blockers, serine/threonine kinases, phosphotidyl inositol-3 kinases, myo-inositol signaling, and Ras oncogenes, inculding growth factor receptors include, for example, epidermal growth factor receptor (EGFr), platelet derived growth factor receptor (PDGFr), erbB2, erbB4, ret, vascular endothelial growth factor receptor (VEGFr), tyrosine kinase with immunoglobulin-like and epidermal growth factor homology domains (TIE-2), insulin growth factor-I (IGFI) receptor, macrophage colony stimulating factor (cfms), BTK, ckit, cmet, fibroblast growth factor (FGF) receptors, Trk receptors (TrkA, TrkB, and TrkC), ephrin (eph) receptors, and the RET protooncogene. An exemplary signal transduction pathway inhibitor is lapatinib (Tykerb/Tyverb®), a dual EGFR/ErbB2 inhibitor.

Tyrosine kinases, which are not growth factor receptor kinases are termed non-receptor tyrosine kinases. Non-receptor tyrosine kinases useful in the present invention, which are targets or potential targets of anti-cancer drugs, include cSrc, Lck, Fyn, Yes, Jak, cAbl, FAK (Focal adhesion kinase), Brutons tyrosine kinase, and Bcr-Abl. Such non-receptor kinases and agents which inhibit non-receptor tyrosine kinase function are described in Sinh, S. and Corey, S. J., (1999) Journal of Hematotherapy and Stem Cell Research 8 (5): 465-80; and Bolen, J. B., Brugge, J. S., (1997) Annual review of Immunology. 15: 371-404.

SH2/SH3 domain blockers are agents that disrupt SH2 or SH3 domain binding in a variety of enzymes or adaptor proteins including, PI3-K p85 subunit, Src family kinases, adaptor molecules (Shc, Crk, Nck, Grb2) and Ras-GAP. SH2/SH3 domains as targets for anti-cancer drugs are discussed in Smithgall, T. E. (1995), Journal of Pharmacological and Toxicological Methods. 34(3) 125-32.

Inhibitors of Serine/Threonine Kinases including MAP kinase cascade blockers which include blockers of Raf kinases (rafk), Mitogen or Extracellular Regulated Kinase (MEKs), and Extracellular Regulated Kinases (ERKs); and Protein kinase C family member blockers including blockers of PKCs (alpha, beta, gamma, epsilon, mu, lambda, iota, zeta). IkB kinase family (IKKa, IKKb), PKB family kinases, akt kinase family members, and TGF beta receptor kinases. Such Serine/Threonine kinases and inhibitors thereof are described in Yamamoto, T., Taya, S., Kaibuchi, K., (1999), Journal of Biochemistry. 126 (5) 799-803; Brodt, P, Samani, A., and Navab, R. (2000), Biochemical Pharmacology, 60. 1101-1107; Massague, J., Weis-Garcia, F. (1996) Cancer Surveys. 27:41-64; Philip, P. A., and Harris, A. L. (1995), Cancer Treatment and Research. 78: 3-27, Lackey, K. et al Bioorganic and Medicinal Chemistry Letters, (10), 2000, 223-226; U.S. Pat. No. 6,268,391; and Martinez-Iacaci, L., et al, Int. J. Cancer (2000), 88(1), 44-52.

Inhibitors of Phosphotidyl inositol-3 Kinase family members including blockers of PI3-kinase, ATM, DNA-PK, and Ku are also useful in the present invention. Such kinases are discussed in Abraham, R. T. (1996), Current Opinion in Immunology. 8 (3) 412-8; Canman, C. E., Lim, D. S. (1998), Oncogene 17 (25) 3301-3308; Jackson, S. P. (1997), International Journal of Biochemistry and Cell Biology. 29 (7):935-8; and Zhong, H. et al, Cancer res, (2000) 60(6), 1541-1545.

Also useful in the present invention are Myo-inositol signaling inhibitors such as phospholipase C blockers and Myoinositol analogues. Such signal inhibitors are described in Powis, G., and Kozikowski A., (1994) New Molecular Targets for Cancer Chemotherapy ed., Paul Workman and David Kerr, CRC press 1994, London.

Another group of signal transduction pathway inhibitors are inhibitors of Ras Oncogene. Such inhibitors include inhibitors of farnesyltransferase, geranyl-geranyl transferase, and CAAX proteases as well as anti-sense oligonucleotides, ribozymes and immunotherapy. Such inhibitors have been shown to block ras activation in cells containing wild type mutant ras, thereby acting as antiproliferation agents. Ras oncogene inhibition is discussed in Scharovsky, O. G., Rozados, V. R., Gervasoni, S. I. Matar, P. (2000), Journal of Biomedical Science. 7(4) 292-8; Ashby, M. N. (1998), Current Opinion in Lipidology. 9 (2) 99-102; and BioChim. Biophys. Acta, (19899) 1423(3):19-30.

As mentioned above, antibody antagonists to receptor kinase ligand binding may also serve as signal transduction inhibitors. This group of signal transduction pathway inhibitors includes the use of humanized antibodies to the extracellular ligand binding domain of receptor tyrosine kinases. For example Imclone C225 EGFR specific antibody (see Green, M. G. et al, Monoclonal Antibody Therapy for Solid Tumors, Cancer Treat. Rev., (2000), 26(4), 269-286); Herceptin® erbB2 antibody (see Tyrosine Kinase Signalling in Breast cancer:erbB Family Receptor Tyrosine Kinases, Breast cancer Res., 2000, 2(3), 176-183); and 2CB VEGFR2 specific antibody (see Brekken, R. A. et al, Selective Inhibition of VEGFR2 Activity by a monoclonal Anti-VEGF antibody blocks tumor growth in mice, Cancer Res. (2000) 60, 5117-5124).

Anti-angiogenic agents including non-receptorMEKngiogenesis inhibitors may alo be useful. Anti-angiogenic agents such as those which inhibit the effects of vascular edothelial growth factor, (for example the anti-vascular endothelial cell growth factor antibody bevacizumab [Avastin™], and compounds that work by other mechanisms (for example linomide, inhibitors of integrin αvβ3 function, endostatin and angiostatin);

Agents used in immunotherapeutic regimens may also be useful in combination with the compounds of invention. Immunotherapy approaches, including for example ex-vivo and in-vivo approaches to increase the immunogenecity of patient tumour cells, such as transfection with cytokines such as interleukin 2, interleukin 4 or granulocyte-macrophage colony stimulating factor, approaches to decrease T-cell anergy, approaches using transfected immune cells such as cytokine-transfected dendritic cells, approaches using cytokine-transfected tumour cell lines and approaches using anti-idiotypic antibodies

Agents used in proapoptotic regimens (e.g., bcl-2 antisense oligonucleotides) may also be used in the combination of the present invention.

Cell cycle signalling inhibitors, including CDK2, CDK4, and CDK6 and inhibitors for the same are described in, for instance, Rosania et al, Exp. Opin. Ther. Patents (2000) 10(2):215-230.

### Adjuvants

When the term “adjuvant” is used in this specification to refer to a component of the immunotherapy, it refers to a substance that is administered in conjunction with the immunotherapy to boost the patient's immune response to the immunogenic component of the immunotherapy (see, e.g., WO 02/32450). This is to be distinguished from an “adjuvant therapy”, which as discussed above is an additional treatment given after the primary treatment for a cancer. Thus an immunotherapy may be an adjuvant treatment; the immunotherapeutic composition may comprise an adjuvant compound, such as those discussed below. Such adjuvants are well known in the art and can be administered in a separate formulation or may be a component of the formulation comprising the immunogenic component of the immunotherapy. Thus the immunotherapeutics as described herein may further comprise a vaccine adjuvant, and/or an immunostimulatory cytokine or chemokine.

Suitable vaccine adjuvants for use in the present invention are commercially available such as, for example, Freund's Incomplete Adjuvant and Complete Adjuvant (Difco Laboratories, Detroit, Mich.); Merck Adjuvant 65 (Merck and Company, Inc., Rahway, N.J.); AS02 (an Adjuvant System containing MPL and QS21 in an oil-in-water emulsion; SmithKline Beecham, Philadelphia, Pa.); AS15 (an Adjuvant System containing MPL, QS21, CpG and liposome); aluminium salts such as aluminium hydroxide gel (alum) or aluminium phosphate; salts of calcium, iron or zinc; an insoluble suspension of acylated tyrosine; acylated sugars; cationically or anionically derivatised polysaccharides; polyphosphazenes; biodegradable microspheres; monophosphoryl lipid A and quil A. Cytokines, such as GM-CSF or interleukin-2, -7, or -12, and chemokines may also be used as adjuvants.

In formulations of the invention it may be desirable that the adjuvant composition induces an immune response predominantly of the Th1 type. High levels of Th1-type cytokines (e.g., IFN-γ, TNFα, IL-2 and IL-12) tend to favour the induction of cell mediated immune responses to an administered antigen. According to one embodiment, in which a response is predominantly Th1-type, the level of Th1-type cytokines will increase to a greater extent than the level of Th2-type cytokines. The levels of these cytokines may be readily assessed using standard assays. For a review of the families of cytokines, see Mosmann and Coffman, Ann. Rev. Immunol. 7:145-173, 1989.

Accordingly, suitable adjuvants that may be used to elicit a predominantly Th1-type response include, for example a combination of monophosphoryl lipid A (MPL), such as 3-O-desacyl-4′-monophosphoryl lipid A together with an aluminium salt. MPL or other toll like receptor 4 (TLR4) ligands such as aminoalkyl glucosaminide phosphates (AGPs) as disclosed in WO9850399, WO0134617 and WO03065806 may also be used alone to generate a predominantly Th1-type response.

Other known adjuvants, which may preferentially induce a TH1 type immune response, include TLR9 antagonists such as synthetic oligodeoxynucleotides (ODNs) containing unmethylated CpG motifs (CpGs or CpG-containing oligodeoxynucleotides). Such oligonucleotides are well known and are described in, for example WO 96/02555.

CpG-containing oligodeoxynucleotides may also be used alone or in combination with other adjuvants. For example, an enhanced system involves the combination of a CpG-containing oligodeoxynucleotide and a saponin derivative particularly the combination of CpG and QS21(Quillaja Saponaria Molina, fraction 21; Antigenics, New York, N.Y., USA) as disclosed in WO00/09159 and WO00/62800.

The formulation may additionally comprise an oil in water emulsion and/or tocopherol.

Another suitable adjuvant is a saponin, for example QS21, that may be used alone or in combination with other adjuvants. For example, an enhanced system involves the combination of a monophosphoryl lipid A and saponin derivative, such as the combination of QS21 and MPL as described in WO 94/00153, or a less reactogenic composition where the QS21 is quenched with cholesterol, as described in WO 96/33739. Other suitable formulations comprise an oil-in-water emulsion and α-tocopherol. A particularly potent adjuvant formulation involving QS21, MPL and α-tocopherol in an oil-in-water emulsion is described in WO 95/17210.

In another embodiment, the adjuvants may be formulated in a liposomal composition.

The amount of MPL used is generally small, but depending on the Immunotherapeutic formulation may be in the region of 1-1000 μg per dose, 1-500 μg per dose, or between 1 to 100 μg per dose.

In an embodiment, the adjuvant system comprises three immunostimulants: a CpG-containing oligonucleotide, MPL, & QS21 either presented in a liposomal formulation or an oil in water emulsion such as described in WO 95/17210.

The amount of CpG-containing oligodeoxynucleotide or immunostimulatory oligonucleotides in the adjuvants or immunotherapeutics of the present invention is generally small, but depending on the immunotherapeutic formulation may be in the region of 1-1000 μg per dose, between 1-500 μg per dose, or between 1 to 100 μg per dose.

The amount of saponin for use in the adjuvants of the present invention may be in the region of 1-1000 μg per dose, between 1-500 μg per dose, between 1-250 μg per dose, or between 1 to 100 μg per dose.

Generally, each human dose may comprise from 1 to 1000 μg of protein antigen. In one embodiment, the dose may comprise 30-300 μg of protein antigen. Useful dosages for a particular immunotherapeutic, and/or for treating a particular tumor type, can be ascertained by standard studies involving observation of appropriate immune responses in vaccinated subjects. Following an initial vaccination, subjects may receive one or several booster immunisation adequately spaced.

In one embodiment, the adjuvant may comprise one or more of MPL, QS21 and an immunostimulatory CpG-containing oligodeoxynucleotide. In an embodiment all three immunostimulants are present. In another embodiment MPL and Qs21 are presented in an oil in water emulsion, and in the absence of a CpG-containing oligodeoxynucleotide.

### Experimentals

### Prevalence of BRAF V600 Mutation and MAGE Expression in Melanoma

DNA of 53 fresh tumor tissues in RNAlater™ were collected during clinical studies. The DNA was extracted with the Maxwell Tissue DNA purification kit (Promega) and quantified by spectrometry. The BRAF mutational status was then tested with an allele-specific PCR assay developed by Response Genetics (RGI). Briefly, a control assay was used to assess the total DNA in the samples by amplifying a polymorphism-free region of exon 13 in the BRAF gene. In parallel, three BRAF variant-specific mutation assays developed to specifically detect the BRAF V600E, V600K and V600D* variants were used to determine the BRAF mutational status. A sample was considered as:

BRAF V600E mutated if the delta Ct [(BRAF V600E Ct)−(BRAF Ex13 Ct)] was inferior to 6.5;
BRAF V600K mutated if the delta Ct [(BRAF V600K Ct)−(BRAF Ex13 Ct)] was inferior to 6.5;
BRAF V600D mutated if the delta Ct [(BRAF V600D Ct)−(BRAF Ex13 Ct)] was inferior to 6.7;
BRAF wild-type if the three aforesaid delta Ct were superior to their respective cut-off.

The MAGEA3 expression level was determined by qRTPCR on the same biopsies.

Table 1 shows the resultant MAGEA3 expression status and BRAF genotype

TABLE 1

| | N = 53 samples | | |
|---|---|---|---|
| | | | % BRAF mutated |
| | | | according to |
| | BRAF genotype | | MAGE-A3 |
| MAGE-A3 expression status | V600E/K/D | WT | expression status |
| MAGE-A3 positive | 24.5% | 30.5% | 44.8% |
| | (13/53) | (16/53) | (13/29) |
| MAGE-A3 negative | 22.5% | 22.5% | 50.0% |
| | (12/53) | (12/53) | (12/24) |

### No Deleterious Effect of BRAF Inhibitor or MEK Inhibitor on MAGE Immunotherapy in Mice

7 groups of 22 CB6F1 female mice received as follows—1) PBS, 2)MAGE-A3/AS15, 3) vehicle, 4) BRAF inhibitor. (GSK436), 5) MEK inhibitor. (GSK212), 6) combo ASCI+GSK 436, and 7) combo ASCI+GSK 212. The MAGE-A3/AS15 was given Intra Muscularly (IM) at day 0 and 14. The B-RAF and MEK inhibitors were given by Intra Gastric gavages (IG) daily from day 0 to day 14.

Two weeks after the second immunization (day 28), the CD4+ and CD8+ T cell responses were analysed on spleen cells of 12 mice per group (4 pools of 3 mice)=primary endpoint, and the antibody response was measured on the 12 mice per group.

T-Cell Responses

The analysis of the T cell response was considered as the primary endpoint and the experiment was powered accordingly. The assay was performed 2 weeks after the last immunization on spleen cells from 12 mice/group (4 pools of 3 mice) immunized twice at 2 weeks interval with the MAGE-A3 ASCI and consists in intracellular cytokine staining by flow cytometry (ICS), assessing the percentage of either CD4+ or CD8+ T cells able to produce cytokines (IFNg and/or TNFa).

Spleen cells of immunized animals were re-stimulated for 2 hrs at 37° C. in 96W plates either with medium (no stimulation) or with a pool of 57, 15mer peptides overlapping by 10AA, covering the entire sequence of MAGE-A3 (1 μg/ml/peptide) in a final volume of 200 μl of RPMI 5% FCS containing co-stimulatory antibodies: anti CD49d and anti CD28 at 2 μg/ml each. After the incubation, the secretion of cytokines was blocked by the addition of 50 μl of brefeldin (1/1000) in RPMI 5% FCS.

Cells were then transferred in a 96W (conic wells) plate, centrifuged (1000 rpm 5′ at 4° C.), washed with 250 μl FACS buffer (PBS 1% FCS). The cell pellets were incubated with 50 μl of 2.4G2 diluted 1/50× in FACS buffer during 10′ at 4° C. to block aspecific binding to Fc receptors. CD4+ and CD8+ T cells were stained (30′ at 4° C.). by addition of 50 μl of Master mix containing fluorescent antibodies (CD4-PE mAb: 1/100 and CD8PerCP mAb: 6/100) in FACS buffer.

Cells were washed in FACS buffer, centrifuged (1200RPM-10′). 200 μl of cytoFix-cytoPerm™ solution were added during 20′ at 4° C., cells were centrifuged. A permeabilizing solution (permWASH™) 1× concentrated in sterile water, was added and cells were centrifuged (1000RPM-5′). Pellets were incubated 2 hrs at 4° C. with 50 μl of a mix of fluorescent antibodies against IFNg APC (mAb: 1/50) and TNFa FITC (mAb: 1/50) in the permWASH™ 1× solution. Cells were washed, centrifuged (1000RPM) and resuspended in FACS buffer before FACS analysis (LSR2 from Becton Dickinson).

After gating on living T cells (1), a total of around 20000 CD4+ T cells and around 10000 CD8+ T cells were acquired (2) and the data expressed as the percentages of these CD4+ or CD8+ T cells which produce cytokines (3).

Data obtained with the non-stimulated cells (medium) are subtracted from the data obtained with the cells stimulated with the MAGE-A3 peptides. Background levels are generally undetectable or very low comprised between 0.01-0.05% in the control group of mice receiving phosphate buffer saline (PBS). Depending on the background, percentages of 0.1 can be considered as positive responses.

To achieve a normal distribution of the response and homogeneity of the variance, the data were first transformed on the log 10 basis. A one-way ANOVA and a Multiple Comparisons (Tukey test) was applied to reveal significant differences between groups.

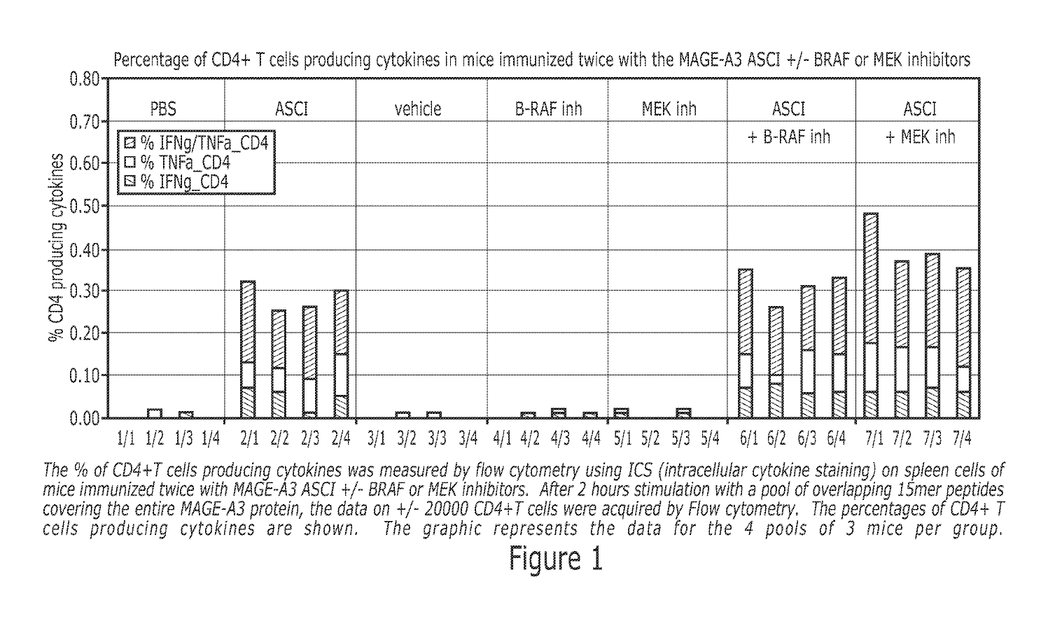

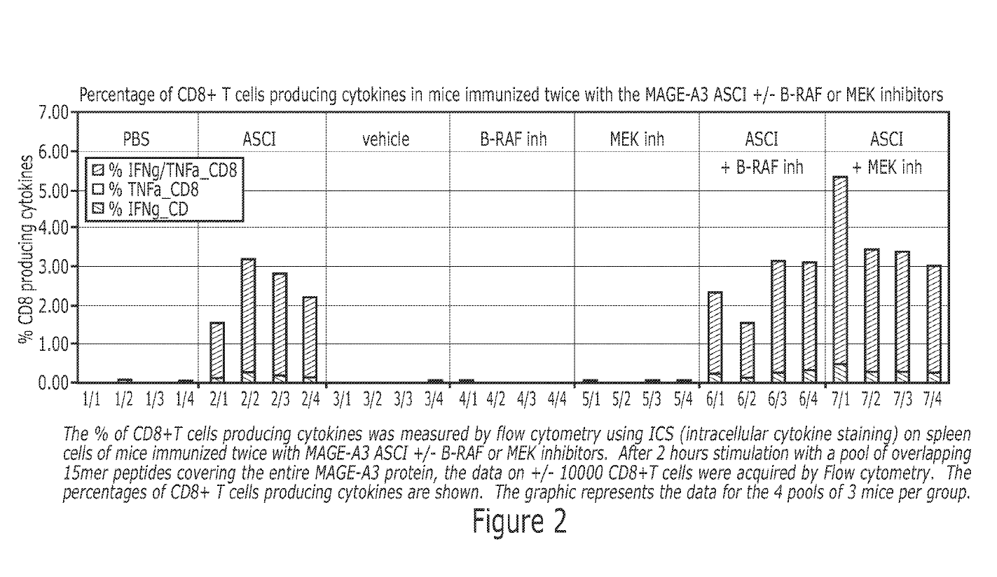

14 days after 2 immunizations, the T cell response was measured on spleen cells of immunized animals using the intracellular cytokine staining assay. The data were analysed as 4 pools of 3 mice per group. The CD4+ T cell response is shown in FIG. 1. The CD8+ T cell response is shown in FIG. 2.

For CD4+ T cell response, the geometric mean ratio between ASCI and ASCI +B-RAF inhibitor was 0.9 with a 95% CI of [0.7-1.16], entirely contained in [0.3-3], and the geometric mean ratio between ASCI and ASCI+MEK inhibitor. was 0.71 with a 95% CI of [0.55-0.92], entirely contained in [0.3-3].

For CD8+ T cell response, the geometric mean ratio between ASCI and ASCI +B-RAF inhibitor. was 0.97 with a 95% CI of [0.53-1.78], entirely contained in [0.3-3]. The geometric mean ratio between ASCI and ASCI+MEK inhibitor. was 0.64 with a 95% CI of [0.35-1.16], entirely contained in [0.3-3]

Antibody Response

The antibody response was evaluated 2 weeks after the last immunization on 12 mice per group. Mice sera were tested by ELISA for the presence of MAGE-A3-specific antibodies 14 days post 2 immunizations.

Before addition of sera the immunoplate was coated overnight at 4° C. with the Mage3 antigen produced in baculovirus. After reaction with the sera for 90′ at 37° C., a biotinylated sheep whole antibody against mouse immunoglobulins was added for 90′ at 37° C. The antigen-antibody complex was revealed by incubation with a streptavidin-biotinylated peroxydase complex for 30′ at 37° C. This complex was then revealed by the addition of tetramethyl benzidine (TMB) for 10′ at Room Temperature and the reaction was stopped with 0.2 M H2SO4. Optical densities were recorded at 450 nm.

Individual mice anti-MAGE-A3 titres were calculated by referring to a standard curve established with a Standard serum (a pool of sera from mice immunized with the MAGE-A3 ASCI-LIMS 20100152) and average calculated for each group.

To achieve a normal distribution of the response and homogeneity of the variance, the data were first transformed on the log 10 basis. A One-way ANOVA and a Multiple Comparisons (Tukey test) was applied to reveal significant differences between groups.

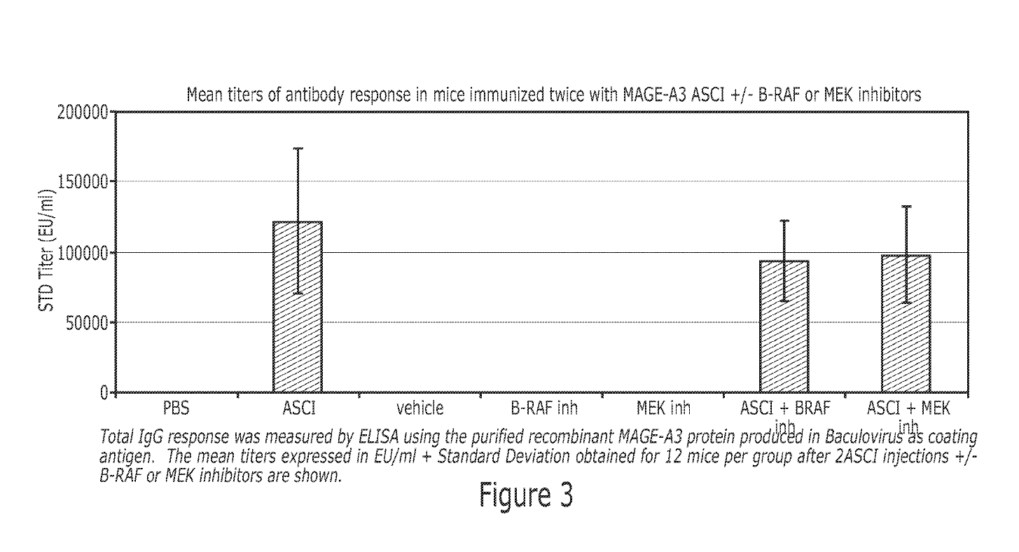

The antibody response was measured by ELISA using a purified recombinant MAGE-A3 protein produced in the Baculovirus expression system as coating antigen. Sera from 12 mice per group were tested individually. FIG. 3 represents the mean titers+/−Standard Deviation of the 12 mice per group.

The geometric mean ratio of the antibody titers between ASCI and ASCI +B-RAF inh. was 1.08 with a 95% CI of [0.91-1.3], entirely contained in [0.3-3]. The geometric mean ratio between ASCI and ASCI+MEK inh. was 1.07 with a 95% CI of [0.9-1.3], entirely contained in [0.3-3].

No Impairment of Overall Immune Competency in Cancer Patients Treated with BRAF Inhibitor

13 patients with tumors carrying a BRAF mutation who underwent treatment with GSK2118436 (the compound of formula (I)), a V600 mutant BRAF specific inhibitor were studied to assess effects of mutant BRAF inhibition on systemic immunity. Peripheral blood immune-monitoring was carried out before and following one or two 28-day cycles of treatment.

As a result, GSK2118436 treatment had no detectable impact on most immune parameters tested, including serum cytokine levels, peripheral blood cell counts, leukocyte subset frequencies, and memory CD4+ and CD8+ T-cell recall responses. A slight increase in serum TNF-α over the course of treatment was observed. In addition, three of the four human leukocyte antigen-A2-positive patients experienced a modest increase in circulating tumor antigen-specific CD8+ T cells following BRAF(V600) inhibitor therapy.

As a conclusion, GSK2118436 treatment resulted in no detectable negative impact on existing systemic immunity or the de novo generation of tumor-specific T cells.

A more detailed report of the underlying analysis and results is provided in Hong D S, Vence L, Falchook G, Radvanyi L G, Liu C, Goodman V, et al. BRAF(V600) Inhibitor GSK2118436 Targeted Inhibition of Mutant BRAF in Cancer Patients Does Not Impair Overall Immune Competency. Clinical cancer research: an official journal of the American Association for Cancer Research. 2012; 18:2326-35, incorporated herein by reference.

What is claimed is:

1. A method for treating a susceptible cancer, in a human in need thereof, said method comprising administering a therapeutically effective amount of
(i) a MAGE-A3 immunotherapeutic, and one or both of
(a) a compound of formula (I) 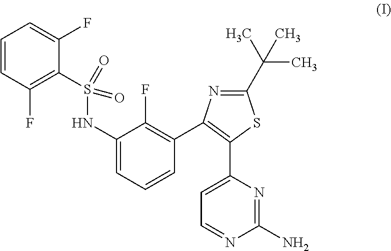 [SMILES: CC(C)(C)c1nc(-c2cccc(NS(=O)(=O)c3c(F)cccc3F)c2F)c(-c2ccnc(N)n2)s1]
or a pharmaceutically acceptable salt thereof, and
(b) a compound of Structure (II) 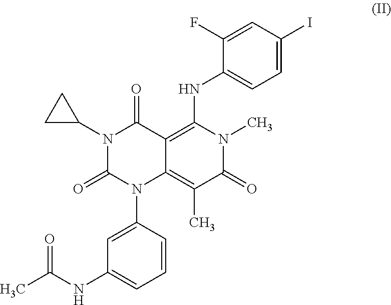 [SMILES: CC(=O)Nc1cccc(-n2c(=O)n(C3CC3)c(=O)c3c(Nc4ccc(I)cc4F)n(C)c(=O)c(C)c32)c1]
or a pharmaceutically acceptable salt or solvate thereof.

2. The method of claim 1, wherein the compound of formula (I) or pharmaceutically acceptable salt thereof is in the form of the methanesulfonate salt.

3. The method of claim 1, wherein the compound of structure (I) is in the form of the dimethyl sulfoxide solvate.

4. The method of claim 1, wherein the MAGE-A3 immunotherapeutic is a Protein D-MAGE-A3 fusion protein.

5. The method of claim 1, wherein the MAGE-A3 immunotherapeutic is a fusion protein comprising SEQ ID NO:2.

6. The method of claim 1 wherein the MAGE-A3 immunotherapeutic comprises an adjuvant.

7. The method of claim 1, wherein the compound of formula (I) and/or (II) or pharmaceutically acceptable salt thereof further comprises a pharmaceutically acceptable diluent or carrier.

8. The method of claim 1, wherein the cancer is selected from head and neck cancer, breast cancer, lung cancer, colon cancer, ovarian cancer, prostate cancer, non-small cell lung carcinoma (NSCLC), gliomas, glioblastoma, astrocytomas, glioblastoma multiforme, Bannayan-Zonana syndrome, Cowden disease, Lhermitte-Duclos disease, inflammatory breast cancer, Wilm's tumor, Ewing's sarcoma, Rhabdomyosarcoma, ependymoma, medulloblastoma, kidney cancer, liver cancer, melanoma, pancreatic cancer, sarcoma, osteosarcoma, giant cell tumor of bone, thyroid cancer, lymphoblastic T cell leukemia, Chronic myelogenous leukemia, Chronic lymphocytic leukemia, Hairy-cell leukemia, acute lymphoblastic leukemia, acute myelogenous leukemia, AML, Chronic neutrophilic leukemia, Acute lymphoblastic T cell leukemia, plasmacytoma, Immunoblastic large cell leukemia, Mantle cell leukemia, Multiple myeloma Megakaryoblastic leukemia, multiple myeloma, acute megakaryocytic leukemia, promyelocytic leukemia, erythroleukemia, malignant lymphoma, hodgkins lymphoma, non-hodgkins lymphoma, lymphoblastic T cell lymphoma, Burkitt's lymphoma, follicular lymphoma, neuroblastoma, bladder cancer, urothelial cancer, vulval cancer, cervical cancer, endometrial cancer, renal cancer, mesothelioma, esophageal cancer, salivary gland cancer, hepatocellular cancer, gastric cancer, nasopharangeal cancer, buccal cancer, cancer of the mouth, GIST (gastrointestinal stromal tumor), and testicular cancer.

9. The method of claim 8, wherein the cancer is melanoma.
